(19) United States
(12) Patent Application Publication
(10) Pub. No.: US 2002/0147155 A1
(43) Pub. Date: Oct. 10, 2002

(54) Prevention and treatment of endometriosis with aryl hydrocarbon receptor binding ligands

(76) Inventors: Warren G. Foster, Ontario (CA); Sanjay Agarwal, Los Angeles, CA (US); Claude L. Hughes JR., Mebane, NC (US)

Correspondence Address: SIDLEY & AUSTIN, 555 West Fifth Street, Los Angeles, CA, 90013-1010, US

(21) Appl. No.: 09/827,398

(22) Filed: Apr. 6, 2001

(51) Int. Cl. A61K 31/7024; A61K 31/405; A61K 31/404

(52) U.S. Cl. 514/23; 514/415; 514/419

(57) ABSTRACT

Disclosed are methods of treating endometriosis in a woman or of preventing endometriosis in a woman at higher than normal risk of developing or suffering recurrence of endometriosis. The inventive methods involve administering to the woman a pharmaceutically acceptable composition containing an aryl hydrocarbon receptor binding ligand, such as indole-3-carbinol, indole-4-carbinol, indole-5-carbinol, diindolylmethane, tryptophan, tryptamine, indole acetic acid, or glucosinolate compounds.

### BACKGROUND OF THE INVENTION

Throughout the application various publications are referenced in parentheses. The disclosures of these publications in their entireties are hereby incorporated by reference in the application in order to more fully describe the state of the art to which this invention pertains.

1. Field of the Invention

This invention relates to the medical arts. In particular, it relates to a method for preventing and treating endometriosis.

2. Discussion of the Related Art

In the course of the normal menstrual cycle, the lining of the uterus, i.e. the endometrium, responds to hormonal regulation by thickening under the influence of estrogen in the proliferative phase of the menstrual cycle and regressing when the hormonal support is withdrawn at the end of the menstrual cycle. Endometriosis is a disease in which abnormal formations of endometriotic tissue develop in locations other than the uterus. Endometriotic tissue resembles endometrium and responds to estrogen by thickening. The presence of endometriotic tissue outside of the uterus is associated with symptoms of infertility and pelvic pain. (Lessey, Medical management of endometriosis and infertility, Fertil. Steril. 73(6):1089-96[2000]; Fedele et al., Pain symptoms associated with endometriosis, Obstet Gynecol 79(5 pt 1):767-69 [1992]). However, the diagnosis of endometriosis generally requires surgical means to identify the abnormal formations. (Farquhar, Endometriosis, BMJ 320(7247):1449-52 [2000]).

Endometriosis is estimated to affect 3% to 18% of women. It is the single most common gynecologic diagnosis responsible for the hospitalization of women aged 15-44 and is found in 53% of adolescents with pelvic pain severe enough to warrant surgical evaluation. (Wheeler, Epidemiology and prevalence of endometriosis, Infertil. Reprod. Med. Clin. N. Amer. 3:545 [1992]; Muse et al., Endometriosis, in Current Obstetric & Gynecologic Diagnosis & Treatment, 801 [DeCherny et al. eds., 1994]).

The etiology of endometriosis has been attributed alternatively to coelomic metaplasia, vascular transport and retrograde menstruation. (Matsuura et al., Coelomic metaplasia theory of endometriosis: evidence from in vivo studies and an in vitro experimental model, Gynecol. Obstet. Invest. 47(Supp 1): 18-20 [1999]; Ueki, Histologic study of endometriosis and examination of lymphatic drainage in and from the uterus, Am. J. Obstet. Gynecol. 165(1):201-9 [1991]; Vinatier et al., The mechanisms of endometriosis, Rev. Prat. 49(3):254-57 [1999]). For retrograde menstruation, a widely accepted etiological mechanism, viable fragments of endometrium flow into the pelvic cavity through the uterine tubes during menstruation. The importance of this mechanism in the etiology of endometriosis is supported by the observation that endometriosis occurs more often in women with outflow defects of the uterine cavity. (Ugur et al., Endometriosis in association with mullerian anomalies, Gynecol. Obstet. Invest. 40(4):261-64 [1995]). Also, partial obstruction of the cervical os in baboons resulted in endometriosis in all of the baboons within three months. (D'Hooghe et al., Development of a model of retrograde menstruation in baboons, Fertil. Steril. 62(3):635-38 [1994]). However, retrograde menstruation, which occurs in 70% to 90% of women, is much more common than endometriosis. (Halme et al., Retrograde menstruation in healthy women and in patients with endometriosis, Obstet. Gynecol. 64(2):151-54 [1984]). Therefore, factors other than access of endometrial contents to the pelvis via retrograde menstruation are thought to contribute to the development and progression of endometriosis.

Endometrial cells destined to become endometriotic implants are different from normal endometrial cells. Endometrial cells from women with endometriosis survived transplantation in athymic nude mice much longer than normal proliferative phase endometrium from women without endometriosis, implying that these two sets of cells are functionally distinct. (Zamah et al., Transplantation of normal and ectopic human endometrial tissue into athymic nude mice, Am. J. Obstet. Gynecol. 149(6):591-97 [1984]). Moreover, cells of endometriotic implants and endometrial cells from women with endometriosis were demonstrated to over-express aromatase, compared to a lack of expression in endometrial cells from women without symptoms of endometriosis and in cells from non-endometriotic pelvic tissues from women with endometriosis. (Noble et al., Aromatase expression in endometriosis, J. Clin. Endocrinol. Metab. 81(1):174-79 [1996]). Furthermore, interleukin-6 and its soluble receptor, which are growth inhibitory for the endometrium are both dysregulated in human endometriotic tissue compared to normal endometrium. (Rier et al., Dysregulation of interleukin-6 responses in ectopic endometrial stromal cells: correlation with decreased soluble receptor levels in peritoneal fluid of women with endometriosis, J. Clin. Endocrinol. Metab. 80(4):1431-37 [1995]). Thus, even while still in the uterus, endometrial cells destined to implant to establish endometriotic lesions are biochemically distinct from their normal counterparts.

Current therapies for endometriosis typically start with a combination of pain management through nonsteroidal anti-inflammatory agents and the creation of a pseudopregnancy state with oral contraceptives. (Muse et al., Endometriosis, in Current Obstetric & Gynecologic Diagnosis & Treatment, 801, 805 [DeCherny et al. eds., 1994]; Luciano et al., Evaluation of oral medroxyprogesterone acetate in the treatment of endometriosis, Obstet. Gynecol. 72(3 pt 1):323-27 [1988]). Pseudopregnancy therapy takes advantage of the similarity between normal endometrial tissue and endometriotic tissue in their responses to changing levels of gonadal steroids. Just as the endometrium thins and regresses during pregnancy, endometriotic tissue seems to regress during pregnancy. Unfortunately, oral estrogen based pseudopregnancy therapy does not relieve pain for many women.

Progestin is also administered to produce a pseudopregnancy state, but it has restrictive side effects including adverse plasma lipoprotein changes, depression, fluid retention, weight gain, breast tenderness and significant breakthrough bleeding. (Vercellini et al., Progestins for symptomatic endometriosis: a critical analysis of the evidence, Fertil. Steril. 68(3):393-401 [1997]). Controlled release devices have been used in combination with synthetic progestins to maintain low concentrations of progestins in an attempt to limit the side effects associated with progestins. (Labrie et al., Controlled release systems and low dose androgens, U.S. Pat. No. 5,434,146).

Another treatment for endometriosis is the creation of a pseudomenopausal state with either Danazol or a gonadotropin-releasing hormone (GnRH) agonist. (Barbieri et al., Danazol in the treatment of endometriosis: analysis of 100 cases with a 4-year follow-up, Fertil. Steril. 37(6): 737-46 [1982]; Vignali, Molecular action of GnRH analogues on ectopic endometrial cells, Gynecol. Obstet. Invest. 45(Supp 1):25 [1998]). This is because endometriosis is widely known to regress after the surgical removal of both ovaries or menopause. (Cook et al., The role of laparoscopy in the treatment of endometriosis, Fertil. Steril. 55(4):663-80 [1991]; Coronado et al., Surgical treatment of symptomatic colorectal endometriosis, Fertil. Steril. 53(3):411-16 [1990]). However, androgenic side effects of Danazol are common and include acne, oily skin, hirsuitism, male pattern hair loss, deepening of the voice, weight gain, edema, and adverse plasma lipoprotein changes. (Barbieri et al., Danazol in the treatment of endometriosis: analysis of 100 cases with a 4-year follow-up, Fertil. Steril. 37(6):737-46 [1982]). Some of these side effects, such as the deepening of the voice are irreversible. In addition, Danazol is expensive, acts as a contraceptive, and may cause virilization of female fetuses in pregnant women. Topical preparations containing Danazol may produce fewer side effects. (Igarashi, Method for treating endometriosis with topical preparations containing Danazol, U.S. Pat. No. 4,997,653).

The side effects of GnRH agonists include hypoestrogenemia-induced bone loss. In addition, the FDA has only approved the use of GnRH agonists for a single six month course. Progestin, Danazol and GnRH agonists all share the common side effects of hot flushes, depression, vaginal dryness and inhibition or contraindication of pregnancy. Lastly, all three agents increase the risk for cardiovascular diseases, osteoporosis, deep vein thrombosis and infertility. (Dodin et al., Bone mass in endometriosis patients treated with GnRH agonist implant or Danazol, Obstet. Gynecol. 77(3):410-15 [1991]; Agarwal et al., Nafarelin vs. Leuprolide acetate depot for endometriosis. Changes in hone mineral density and vasomotor symptoms. Nafarelin Study Group, J. Reprod. Med. 42(7):413-23 [1997]; Agarwal, Comparative effects of GnRH agonist therapy. Review of clinical studies and their implications, J. Reprod. Med. 43(3 Supp):293-98 [1998]).

Alternative treatments for endometriosis with fewer side effects include the use of phytoestrogens. Hughes et al. taught a method using isoflavones, singly or in combination with at least one hormonal therapeutic agent, to treat and/or prevent endometriosis in females. (Hughes et al., Methods of treating or preventing endometriosis with phytoestrogens, U.S. Pat. No. 5,942,539).

Antiestrogens such as benzothiophenes, droloxifene and benzofurans have also been used to treat and/or inhibit endometriosis. (Black et at., Methods of inhibiting endometriosis, U.S. Pat. No. 5,693,656; Thompson, Use of droloxifene for the treatment of prostatic disease, endometriosis and obesity, U.S. Pat. No. 5,852,059; and Fontana, Methods for inhibiting endometriosis, U.S. Pat. No. 5,554,600).

Inhibitors of steroidogenesis, such as aromatase inhibitors, have also been used to treat endometriosis. (Labrie et al., Method of treating prostate adenocarcinoma, adenocarcinoma, prostate benign hypertrophia and endometriosis, U.S. Pat. No. 5,389,613; Fujise et al., Treatment of endometriosis, U.S. Pat. No. 5,166,200; Okada et al., Substituted tertiary amino compound or salt thereof, U.S. Pat. No. 5,538,976; Okada et al., Triazolylated tertiary amine compound or salt thereof, U.S. Pat. No. 5,674,886; Nakakoshi et al., New androstenedione derivative, Japanese Patent No. 6,145,193A; Nakayama et al., Aromatase inhibitor, Japanese Patent No. 7,069,883A; Kimura et al., New compound SNA-60-367S, Japanese Patent No. 7,238,090A; Takayama et al., Treatment of severe postmenopausal endometriosis with an aromatase inhibitor, Fertil. Steril. 69(4):709-13 [1998]).

However, a recent study demonstrated that aromatase is involved in an autocrine positive feedback mechanism which favors the continuous production of estrogen and prostaglandin in endometriotic stromal cells, possibly increasing estrogen-associated cancer risks. (Bulun et al., Estrogen production in endometriosis and use of aromatase inhibitors to treat endometriosis, Endocr. Relat. Cancer 6(2):292-301 [1999]).

The aryl hydrocarbon receptor (AhR) is a ligand-activated transcription factor involved in the regulation of several genes, including some estrogen responsive target genes. (Hahn, The aryl hydrocarbon receptor: a comparative perspective, Comp. Biochem. Physiol. C. Pharmacol. Toxicol. Endocrinol. 121(1-3):23-53 (1998); Safe et al., Ah receptor agonists as endocrine disruptors: antiestrogenic activity and mechanisms, Toxicol. Lett. 28(102-103):343-47 (1998). Aryl hydrocarbon receptor (AhR) is a high affinity low capacity protein that selectively binds chemicals such as 2,3,7,8-Tetrachlorodibenzo-p-dioxin (TCDD) and polynuclear aromatic hydrocarbons. (Okey et al., Regulatory gene product of the Ah locus. Characterization of the cytosolic inducer-receptor complex and evidence for its nuclear translocation, J. Biol. Chem. 254(22): 11636-48 [1979]; Okey et al., Temperature-dependent cytosol-to-nucleus translocation of the Ah receptor for 2,3,7,8-tetrachlorodibenzo-p-dioxin in continuous cell culture lines, J. Biol. Chem. 255(23):11415-22 [1980]; Farrell et al., Synthesis and aryl hydrocarbon receptor binding properties of radiolabeled polychlorinated dibenzofuran congeners, Arch. Biochem. Biophys. 259(1): 185-95 [1987]). AhR is present in many tissues including placenta and various tumor cells including those from breast and cervix. (Manchester et al., Ah receptor in human placenta: stabilization by molybdate and characterization of binding of 2,3,7,8-tetrachlorodibenzo-p-dioxin, 3-methylcholanthrene, and benzo(a)pyrene, Cancer Res. 47(18):4861-8 [1987]; Harris et al, Structure-dependent induction of aryl hydrocarbon hydroxylase in human breast cancer cell lines and characterization of the Ah receptor, Cancer Res. 49(16):4531-5 [1989]; Thomsen et al., Differences in 2,3,7,8-tetrachlorodibenzo-p-dioxin-inducible CYPlAI expression in human breast carcinoma cell lines involve altered trans-acting factors, Eur. J. Biochem. 197(3):577-82 [1991]; Wang et al., Characterization of the aryl hydrocarbon receptor in the human C-4II cervical squamous carcinoma cell line, Biochem. Pharmacol. 43(7):1635-42 [1992]).

In various tissues, aryl hydrocarbon receptor ligand binding has been shown to influence the expression of cytokines, such as epidermal growth factor, interleukin-1, and tumor growth factor. (Madhukar et al., Effects of in vivo-administered 2,3,7,8-tetrachlorodibenzo-p-dioxin on receptor binding of epidermal growth factor in the hepatic plasma membrane of rat, guinea pig, mouse, and hamster, Proc. Natl. Acad. Sci. U.S.A. 81(23):7407-11 [1984]; Sutter et al., Targets for dioxin: genes for plasminogen activator inhibitor-2 and interleukin-1 beta, Science 254(5030):415-18 [1991]; Gaido et al., 2,3,7,8-tetrachlorodibenzo-p-dioxin-dependent regulation of transforming growth factors-alpha and -beta 2 expression in a human keratinocyte cell line involves both transcriptional and post-transcriptional control, J. Biol. Chem. 267(34):24591-95 [1992]).

There are no known endogenous ligands which bind to this receptor, although tryptophan and its metabolites tryptamine and indole acetic acid, have been shown to bind to and activate the aryl hydrocarbon receptor in culture. (Heath-Pagliuso et al., Activation of the Ah receptor by tryptophan and tryptophan metabolites, Biochemistry 37(33):11508-15 [1998]).

While, a recent study concluded that expression of aryl hydrocarbon receptor and dioxin-related genes in the endometrium did not differ in women with or without endometriosis, other studies have implied that aryl hydrocarbon receptor may play a role in endometriosis. (Igarashi et al., Expression of Ah receptor and dioxin-related genes in human uterine endometrium in women with or without endometriosis, Endocr. J. 46(6):765-72 [1999]). Other studies showed that 2,3,7,8-tetrachlorodibenzo-p-dioxin (TCDD), an aryl hydrocarbon receptor agonist promoted endometriosis in rodents and monkeys. (Johnson et al., Promotion of endometriosis in mice by polychlorinated dibenzo-p-dioxins, dibenzofurans, and biphenyls, Environ. Health Perspect. 105(7):750-55 [1997]; Cummings, AM et al., Promotion of endometriosis by 2,3,7,8-tetrachlorodibenzo-p-dioxin in rats and mice: time-dose dependence and species comparison, Toxicol. Appl. Pharmacol. 138:131-39 [1996]; Rier, S E et al., Endometriosis in rhesus monkeys (Macaca mulatta) following chronic exposure to tetrachlorodibenzo-p-dioxin, Fund. Appl. Toxicol. 21:433-41 [1993]). Furthermore, a 15 year rhesus monkey study showed that the incidence of endometriosis was directly correlated with TCDD exposure and that the severity of disease was dose dependent. (Rier et al., Endometriosis in rhesus monkeys following chronic exposure to 2,3,7,8-tetrachlorodibenzo-p-^{dioxin,} Fundam. Appl. Toxicol. 21(4):433-41 [1993]).

A connection may exist between the aryl hydrocarbon receptor and reduced levels of estrogen. Oral administration of indole-3-carbinol, another aryl hydrocarbon receptor ligand, has been shown to reduce levels of estrogen metabolites in women, implying that oral indole-3-carbinol can reduce activation of the estrogen receptor. (Michnovicz et al., Changes in levels of urinary estrogen metabolites after oral indole-3-carbinol treatment in humans, J. Natl. Cancer Inst. 89(10):718-23 [1997]). Two studies have shown that indole-3-carbinol alters estrogen metabolism toward 2-hydroxylation of estrogen, thereby decreasing activation of the estrogen receptor. (Michnovicz et al., Altered estrogen metabolism and excretion in humans following consumption of indole-3-carbinol, Nutr. Cancer 16(1):59-66 [1991]; Michnovicz et al., Changes in levels of urinary estrogen metabolites after oral indole-3-carbinol treatment in humans, J. Natl. Cancer Inst. 89(10):718-23 [1997]).

Indole-3-carbinol and diindolylmethane have been used to treat estrogen related diseases. Safe disclosed compounds and compositions of substituted indole-3-carbinols and diindolylmethanes that are suitable for treating estrogen-dependent tumors. (Safe, Indole-3-Carbinol, Diindolylmethane and Substituted Analogs as Antiestrogens, U.S. Pat. No. 5,945,808; Safe, Indole-3-Carbinol, Diindolylmethane and Substituted Analogs as Antiestrogens, International Application No. PCT/US98/04669). However, Safe disclosed an animal experiment that showed that both diindolylmethane and 5-fluoro-diindolylmethane caused an increase in uterine wet weight as a percentage of body weight.

Accordingly, there exists a need for an effective method for treating or preventing endometriosis without the serious side effects of the currently available treatments. This and other benefits are provided by the present invention.

### SUMMARY OF THE INVENTION

The present invention relates to a method of treating endometriosis in a woman diagnosed as having endometriosis. The method involves administering to the woman having endometriosis a pharmaceutically acceptable composition comprising at least one aryl hydrocarbon receptor (AhR) binding ligand, in an amount effective to reduce the size of endometriotic tissue in the woman.

The present invention also relates to a method of preventing endometriosis in a woman at higher than normal risk of developing or suffering recurrence of endometriosis. The method involves administering to the woman a pharmaceutically acceptable composition comprising at least one aryl hydrocarbon receptor binding ligand, in an amount effective to reduce or prevent the growth or thickening of endometriotic tissue in the woman.

By benefit of the inventive methods, the symptoms of endometriosis are prevented or mitigated.

The present invention also relates to a kit containing a pharmaceutically acceptable composition that includes an amount of at least one aryl hydrocarbon receptor binding ligand and instructions for its use in treating or preventing endometriosis. The kit is useful for practicing the inventive methods.

The present invention also relates to the use of an aryl hydrocarbon receptor binding ligand in the manufacture of a medicament for inhibiting the growth of endometriotic tissues and/or reducing the size of endometriotic tissues.

These and other advantages and features of the present invention will be described more fully in a detailed description of the preferred embodiments which follows.

### DETAILED DESCRIPTION OF THE PREFERRED EMBODIMENTS

The present invention is directed to the use of aryl hydrocarbon receptor (AhR) binding ligands to treat or prevent endometriosis. The method involves administering to a woman having endometriosis or to a woman at higher than normal risk of developing endometriosis a dose of at least one aryl hydrocarbon receptor binding ligand in an amount sufficient to reduce the size or inhibit the growth or thickening of endometriotic tissue in the woman, relative to a control minus the aryl hydrocarbon receptor binding ligand under similar physiological conditions, for example, at a given stage in the menstrual cycle.

For the purposes of the present invention, successful treatment of endometriosis in accordance with the inventive method encompasses, but is not limited to, reducing the size of endometriotic tissue present in the woman. In accordance with the inventive method “reducing the size” of endometriotic tissue encompasses reducing the mass or weight, diameter, length, width, circumference, and/or the thickness or height, of the endometriotic tissue. Also included as indicative of successful treatment in accordance with the inventive method are detectable improvement in symptoms of endometriosis, for example, a pregnancy and/or a reduction in pelvic pain experienced by the woman, regardless of whether the size of her endometriotic tissue is actually measured.

For the purposes of the present invention, preventing endometriosis encompasses inhibiting or reducing the size of endometriotic tissue present in the woman and/or preventing the development symptoms of endometriosis, regardless of whether the size of her endometriotic tissue is actually measured.

In accordance with the present invention, a woman is a female human post-menarche, including pubescent and adult women having periodic menses, menopausal women, and postmenopausal women. A woman having endometriosis refers to a woman medically diagnosed with endometriosis. Endometriosis is a condition in which abnormal formations of endometriotic tissue develop in locations other than the uterus. Endometriotic tissue resembles endometrium and responds to estrogen by thickening. Typically, the diagnosis of endometriosis is done by surgical means, such as laparoscopy or laparotomy, involving direct observation of the endometriotic tissue. However, other medically accepted means of diagnosis are contemplated for purposes of the present invention. Clinical symptoms of endometriosis can also contribute to the diagnosis of the condition. Symptoms commonly include infertility and pelvic pain; low sacral backaches; bloody urine or stool; pain or bleeding with defecation, urination, or intercourse; pelvic discomfort or pressure; and premenstrual spotting.

A woman at higher than normal risk of developing endometriosis is a woman at greater risk than the general population of women of developing endometriosis for the first time or suffering a recurrence of endometriosis. This does not mean that untreated the woman at higher than normal risk of developing endometriosis will certainly develop the condition, merely that her aggregated risk factors are greater than average. Known risk factors for endometriosis include early menarche (before age 13 years), frequent menstruations (e.g., menstrual cycles of 27 days or less), unusually long menstrual period (5-7 days or longer), chronic pelvic pain, especially with stenosis of the external cervical os, advanced age, Asian race, the presence of Mullerian anomalies (e.g., duplicate cervix and vagina), long duration of uninterrupted menstrual cycles, long duration of intrauterine device (IUD) use, infertility, nulliparity, only one live birth, or after ten years since the last birth. (E.g., Brube, S. et al., Characteristics related to the prevalence of minimal or mild endometriosis in infertile women. Canadian Collaborative Group on Endometriosis, Epidemiology 9(5):504-10 [1998]; Barbieri, R. L., Stenosis of the external cervical os: an association with endometriosis in women with chronic pelvic pain, Fertil. Steril. 70(3):571-73 [1998]; Parizinni, F. et al., Pelvic endometriosis: reproductive and menstrual risk factors at different stages in Lombardy, northern Italy, J. Epidemiol. Community Health 49(1):61-64 [1995]; Matorras, R. et al., Epidemiology of endometriosis in infertile women, Fertil. Steril. 63(1):34-38 [1995]; Sangi-Haghpeyker, H. et al., Epidemiology of endometriosis among parous women, Obstet. Gynecol. 85(6):983-92 [1995]; Moen, M. H., Is a long period without childbirth a risk factor for developing endometriosis?, Hum. Reprod. 6(10):1404-07 [1991]; Ketz, M. D. et al., Duplicate cervix and vagina associated with infertility, endometriosis, and chronic pelvic pain, Obstet. Gynecol. 84(4 Pt 2):701-03 [1994]; Vercellini, P. et al., Menstrual characteristics in women with and without endometriosis, Obstet. Gynecol. 90(2):264-68 [1997]; Arumugam, K. and Lim, J. M., Menstrual characteristics associated with endometriosis, Br. J. Obstet. Gynecol. 104(8):948-50 [1997]; Eskenazi, B. and Warner, M. L., Epidemiology of endometriosis, Obstet. Gynecol. Clin. North Am. 24(2):235-58 [1997]; Cramer, D. W. et al., The relation of endometriosis to menstrual characteristics, smoking, and exercise, JAMA 255(14): 1904-08 [1986]).

Women having had cervical conization or gynecological laparotomies, ovarian surgeries, or hysterectomies are also at higher than normal risk for endometriosis. (E.g., Moen, M. H. and Schei, B., Epidemiology of endometriosis in a Norwegian county, Acta Obstet. Gynecol. Scand. 76(6):559-62 [1997]).

Women who have used oral contraceptives are also at higher than normal risk of developing endometriosis. (E.g., Parazzini, F. et al, Oral contraceptive use and risk of endometriosis. Italian Endometriosis Study Group, Br. J. Obstet. Gynaecol. 106(7):695-99 [1999]).

Familial risk factors can also contribute to a higher than normal risk of developing endometriosis, such as a sibling, mother, aunt, or cousin having been diagnosed with endometriosis. (E.g., dos Reis, R. M. et al., Familial risk among patients with endometriosis, J. Assist. Reprod. Genet. 16(9):500-03 [1999]; Treloar, S. A. et al., Genetic influences on endometriosis in an Australian twin sample, Fertil. Steril. 71(4):701-10 [1999]; Hadfield, R. M. et al., Endometriosis in monozygotic twins, Fertil. Steril. 68(5):941-42 [1997]; Moen, M. H., Endometriosis in monozygotic twins, Acta Obstet. Gynecol. Scand. 73(1):59-62 [1994]; Moen, M. H. and Magnus, P., The familial risk of endometriosis, Acta Obstet. Gynecol. Scand. 72(7):560-64 [1993]).

The preceding is merely illustrative of factors that can contribute to a woman being at higher than normal risk of developing endometriosis, and is not an exhaustive list.

The present invention is not committed to any particular mechanism by which the aryl hydrocarbon receptor binding ligand operates to reduce the size of, or inhibit the growth or thickness of, endometriotic tissue.

For purposes of the present invention a useful aryl hydrocarbon receptor binding ligand specifically binds and activates the aryl hydrocarbon receptor (AhR). (E.g., Manchester et al., Ah receptor in human placenta: stabilization by molybdate and characterization of binding of 2,3,7,8-tetrachlorodibenzo-p-dioxin, 3-methylcholanthrene, and benzo(a)pyrene, Cancer Res. 47(18):4861-8 [1987]; Harris et al., Structure-dependent induction of aryl hydrocarbon hydroxylase in human breast cancer cell lines and characterization of the Ah receptor, Cancer Res. 49(16):4531-5 [1989]; Thomsen et al., Differences in 2,3,7,8-tetrachlorodibenzo-p-dioxin-inducible CYPlAI expression in human breast carcinoma cell lines involve altered trans-acting factors, Eur. J. Biochem. 197(3):577-82 [1991]; Wang et al., Characterization of the aryl hydrocarbon receptor in the human C-4II cervical squamous carcinoma cell line, Biochem. Pharmacol. 43(7):1635-42 [1992]). Included among AhR binding ligands are 2,3,7,8-tetrachlorodibenzo-p-dioxin (TCDD), 3-methylcholanthrene, and benzo(a)pyrene.

Useful aryl hydrocarbon receptor (AhR) binding ligands also include, most preferably indole 3-carbinol (I3C), indole-4-carbinol (I4C), indole-5-carbinol (I5C), and diindolylmethane (DIM). Diindolylmethane is a major acid-catalyzed metabolite of indole 3-carbinol that is formed in the mammalian gastrointestinal tract after ingestion of indole 3-carbinol. However, whether or not a particular aryl hydrocarbon receptor binding ligand is metabolized or converted in any way in a human gastrointestinal tract before its entry into the blood stream does not limit the embodiments of aryl hydrocarbon receptor binding ligands that can be used in accordance with the present invention. Other preferred AhR binding ligands include tryptophan, tryptamine, and indole acetic acid.

For purposes of the present invention, AhR binding ligands also include glucosinolate compounds, which are phytochemical components of the Family Cruciferae, particularly members of the genus Brassica (e.g., mustard, broccoli, Brussels sprouts, cabbage, kale, and cauliflower), which are typically released when the plant is crushed or cooked. Glucosinolates are converted in the mammalian digestive system to polyaromatic indole compounds such as indole-3-carbinol, indole-4-carbinol, indole-5-carbinol, and DIM. Examples of useful glucosinolate compounds include glucobrassicin (3-indolylmethyl glucosinolate), sinigrin, progoitrin, gluconapin (3-butenyl glucosinolate), and glucoraphinin (4-methylsulfinylbutyl glucosinolate). Useful glucosinolate compounds also include derivatized glucosinolates and salts thereof, such as, but not limited to, indolyl glucosinolates, for example 4-hydroxyglucobrassicin, or sinalbin (4-hydroxybenzylglucosinolate).

The AhR binding ligands are obtained commercially. Alternatively, they are synthesized from commercially available precursors, and/or purified or isolated from naturally occurring sources by known biochemical means. (E.g., Safe, S, Indole-3-carbinol, diindolylmethane and substituted analogs as antiestrogens, U.S. Pat. No. 5,948,808; Graser, G. et al., The methionine chain elongation pathway in the biosyntheis of glucosinolates in Eruca sativa, Arch. Biochem. Biophys. 378(2):411-19 [2000]; Szmigielska, A. M. et al., Use of Anion-Exchange Membrane Extraction for the High-Performance Liquid Chromatographic Analysis of Mustard Seed Glucosinolates, J. Agric. Food Chem. 48(11):5190-94 [2000]). Synthetic or semi-synthetic versions or derivatives of AhR binding ligands are also useful in the inventive method, as are pharmaceutically acceptable salts of AhR ligand compounds, for example, sodium, magnesium, calcium, hydrochloride, chloride, sulfate, carbonate, or bicarbonate salts. Thus, the AhR binding ligand(s) can be administered, in accordance with the method, as isolated AhR binding ligand(s), which are usefully included in the pharmaceutically acceptable compositions of the present invention, or they may be administered as dietary supplements, for example, as liquid or dried extracts of cruciferous vegetables, seeds and/or herbs, or unextracted, dehydrated cruciferous vegetables, seeds and/or herbs, which can be conveniently ground or powdered.

An effective dose for reducing the size of endometriotic tissue in a woman is an amount sufficient to reduce the size of endometriotic tissue in the woman relative to a control minus the aryl hydrocarbon receptor binding ligand. The effective amount for each woman depend upon the size and individual physiology of the woman. The administered dose of AhR binding ligand should be adjusted as needed, based on prudent periodic monitoring of the woman's condition by the prescribing physician. Preferably, the aryl hydrocarbon receptor binding ligand is delivered in an effective dose of about 1 milligram to about 10 grams per day, and most preferably about 1 gram to about 10 grams per day, which effective dose is provided to the woman in a single daily administration, or divided among two or more administrations per day. The preferred effective dose range of AhR ligands, in accordance with the invention, is well below toxic levels. AhR ligands are known to induce toxic effects at high levels through a variety of mechanisms including induction of liver enzymes, altered thyroid function and immunosuppression. Among the most toxic are 2,3,7,8-tetrachlorodibenzo-p-dioxin, 3-methylcholanthrene, and benzo(a)pyrene, and among the least toxic are the so-called “dietary compounds” found in cruciferous plants, such as the indole carbinols (and derivative DIM) and glucosinolates, requiring exceedingly high doses to induce detectable toxic effects. Hence, the practitioner is advised to avoid doses in excess of about 1-10 g/day for “dietary compounds,” and at least three orders of magnitude less for TCDD, 3-methylcholanthrene, and benzo(a)pyrene.

An effective amount is a dose of AhR binding ligand that produces a reduction in the size of endometriotic tissue in the woman or other detectable improvement in symptoms of endometriosis, for example, a pregnancy and/or a reduction in pelvic pain experienced by the woman, regardless of whether the size of her endometriotic tissue is actually measured. The skilled practitioner will readily apprehend that the tests appropriately employed to determine an effective amount or dose will depend on the individual clinical needs of each patient. The effective amount for each woman will depend upon the physiologic reactions of the patient to whom the pharmaceutically acceptable compositions of the present invention are administered, and the patients reactions will be monitored by the prescribing physician. It is contemplated that the pharmaceutically acceptable compositions of the present invention can be formulated and manufactured at more than one concentration of AhR binding ligand, such that modular increments of AhR binding ligand can be easily administered within the dose range of about 1 milligram to about 10 grams per day. This will give the physician more choices in finding the best effective dose for each patient.

The aryl hydrocarbon receptor binding ligands are administered by any suitable method. Representative methods include giving, providing, feeding, dispensing, inserting, injecting, infusing, perfusing, prescribing, furnishing, treating with, taking, swallowing, eating or applying a pharmaceutically acceptable composition of the present invention. Methods of administering are well known to those of skill in the art and include most preferably oral administration and/or enteral administration.

Useful delivery routes for the administration of the AhR binding ligand include intradermal, subcutaneous, intramuscular, intravenous, intravaginal, intranasal, intrabronchial, and rectal delivery routes. Most preferably the pharmaceutically acceptable composition comprising the AhR binding ligand is administered by an ingestive delivery route.

In embodiments of the method employing administration by an ingestive delivery route, the pharmaceutically acceptable composition is preferably ingested an hour or more after a meal, most preferably when the stomach has cleared the contents of the woman's last meal. However, ingestive administration in the setting of a meal, i.e., along with or substantially simultaneously with the meal is also useful, in accordance with the inventive method.

It is also useful to administer the pharmaceutically acceptable composition by ingestion, when the woman is in a fasted state, particularly if the pharmaceutically acceptable composition containing the aryl hydrocarbon receptor binding ligand is formulated for long acting or extended release. The pharmaceutical industry has developed all sorts of slow and/or sustained-release technologies. Sustained-release formulations employ several methods. The most common is a tablet containing an insoluble core; a drug applied to the outside layer is released soon after the medication is ingested, but drug trapped inside the core is released more slowly. Capsules containing multiparticulate units of drug with coatings that dissolve at different rates are designed to give a sustained-release effect. A preferred embodiment of the present method involves a systemic delivery route, i.e., a route whereby aryl hydrocarbon receptor binding ligands are delivered to body tissues primarily via the blood stream. Entry of aryl hydrocarbon receptor binding ligands into the blood stream of a human can occur by any route, system, device, or medium. For the purposes of the present invention, a systemic delivery route can include an ingestive delivery route, or a parenteral delivery route, for example, a transdermal or transmucosal delivery route. Transmucosal delivery routes include delivery of the AhR binding ligand through the mucosa or epithelium of the mouth including the sublingual epithelium, through the vaginal epithelium, or through the rectal epithelium.

Systemic delivery systems include most conventionally, but are not limited to, ingestive delivery systems, injections, or intravenous drip. A preferred ingestive delivery system is a pharmaceutically acceptable composition formulated as a food additive or food supplement for humans, in the form of an aqueous solution, emulsion, or suspension, or a powder, tablet, microspheres, capsule or caplet.

Other useful systemic delivery systems are known and include, but are not limited to, implant; transmucosal delivery matrices; or suppositories or gels.

In accordance with the invention, pharmaceutically acceptable compositions are formulated to deliver an effective dose of at least one aryl hydrocarbon receptor binding ligand by the above-described or any other pharmaceutically acceptable systemic delivery system, preferably in an amount of about 1 milligram to about 10 grams per day of aryl hydrocarbon receptor binding ligand.

The pharmaceutically acceptable compositions can be formulated for oral or enteral use, for example, as tablets, troches, caplets, microspheres, hard or soft capsules, lozenges, aqueous or oily suspensions, dispersible powders or granules, emulsions, syrups, elixirs or enteral formulas. Compositions intended for oral use are prepared according to any method known to the art for the manufacture of pharmaceutical compositions. Compositions can also be coated by the techniques described in the U.S. Pat. Nos. 4,256,108; 4,160,452; and 4,265,874, to form osmotic therapeutic tablets for controlled release. Other techniques for controlled release compositions, such as those described in the U.S. Pat. Nos. 4,193,985; and 4,690,822; 4,572,833 can be used in the formulation of the inventive pharmaceutically acceptable compositions.

As well as the aryl hydrocarbon receptor binding ligands, the pharmaceutically acceptable composition of the present invention can optionally contain pharmaceutically acceptable solvent(s), adjuvant(s) and/or pharmaceutically acceptable non-medicinal, non-toxic carrier(s), binder(s), thickener(s), and/or filler substance(s) that are known to the skilled artisan for the formulation of tablets, pellets, capsules, solutions, emulsions, suspensions, and any other form suitable for use. The carriers which can be used include glucose, lactose, sucrose, gum acacia, gelatin, mannitol, starch, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes can be used. Also contemplated are additional medicinal or nutritive additives in combination with at least one aryl hydrocarbon receptor binding ligands, as may be desired to suit the more particular needs of the practitioner.

The pharmaceutically acceptable composition can be formulated and manufactured at more than one concentration unit of aryl hydrocarbon receptor binding ligand, such that modular incremental amounts of AhR binding ligands are easily administered. Preferably, the composition is formulated in a delivery system to deliver a dose of about 10 to about 200 mg of aryl hydrocarbon receptor binding ligands. These preferred dose ranges provide beneficial effect with essentially no toxic risk.

A preferred embodiment of the present pharmaceutical composition is formulated for a systemic delivery system such as, but not limited to, ingestive, transmucosal, or injection systems, including for delivery by intradermal, subcutaneous, intramuscular, intravenous, intravaginal, intranasal, intrabronchial, and rectal delivery routes.

A preferred ingestive delivery system is a pharmaceutically acceptable food additive or food supplement for humans, formulated as a powder, tablet, microspheres, capsule or caplet.

Another preferred embodiment of the pharmaceutically acceptable composition of the present invention is a formulation for systemic transmucosal delivery of at least one aryl hydrocarbon receptor binding ligand. A variety of pharmaceutically acceptable systems for transmucosal delivery of therapeutic agents are known in the art and are compatible with the practice of the present invention. (Heiber et al., Transmucosal delivery of macromolecular drugs, U.S. Pat. Nos. 5,346,701 and 5,516,523; Longenecker et al., Transmembrane formulations for dug administration, U.S. Pat. No. 4,994,439). Transmucosal delivery devices may be in free form, such as a cream, gel, or ointment, or may comprise a determinate form such as a tablet, patch, or troche. For example, delivery of at least one aryl hydrocarbon receptor binding ligand may be via a transmucosal delivery system comprising a laminated composite of, for example, an adhesive layer, a backing layer, a permeable membrane defining a reservoir containing at least one aryl hydrocarbon receptor binding ligand, a peel seal disc underlying the membrane, one or more heat seals, and a removable release liner. (Ebert et al, Transdermal delivery system with adhesive overlay and peel seal disc, U.S. Pat. No. 5,662,925; Chang et al., Device for administering an active agent to the skin or mucosa, U.S. Pat. Nos. 4,849,224 and 4,983,395).

Alternatively, a tablet or patch for delivery through the oral mucosa can comprise an inner layer containing the therapeutic agent of choice, a permeation enhancer, such as a bile salt or fusidate, and a hydrophilic polymer, such as hydroxypropyl cellulose, hydroxypropyl methylcellulose, hydroxyethylcellulose, dextran, pectin, polyvinyl pyrrolidone, starch, gelatin, or any of a number of other polymers known to be useful for this purpose. This inner layer can have one surface adapted to contact and adhere to the moist mucosal tissue of the oral cavity and may have an opposing surface adhering to an overlying non-adhesive inert layer. Optionally, such a transmucosal delivery system can be in the form of a bilayer tablet, in which the inner layer also contains additional binding agents, flavoring agents, or fillers. Some useful systems employ a non-ionic detergent along with a permeation enhancer. These examples are merely illustrative of available transmucosal delivery technology and are not limiting of the present invention.

Another preferred embodiment of the pharmaceutically acceptable composition is a gel for systemic delivery of at least one aryl hydrocarbon receptor binding ligand via the rectal or vaginal mucosa, similar to gels commonly used for the delivery of various other therapeutic agents. Hydrogel matrices are known for this purpose. (Feijen, Biodegradable hydrogel matrices for the controlled release of pharmacologically active agents, U.S. Pat. No. 4,925,677). Such biodegradable gel matrices can be formed, for example, by cross-linking a proteinaceous component and a polysaccharide or mucopolysaccharide component, then loading with at least one aryl hydrocarbon receptor binding ligand to be delivered. Other conventional rectal or intravaginal suppository systems are also usefully employed for delivering AhR binding ligands in accordance with the invention.

Another preferred embodiment of the pharmaceutically acceptable composition of the present invention is one formulated for the systemic delivery of at least one aryl hydrocarbon receptor binding ligand via a biodegradable matrix implanted within the body or under the skin of a human or non-human vertebrate. The implant matrix may be a hydrogel similar to those described above. Alternatively, it may be formed from a poly-alpha-amino acid component. (Sidman, Biodegradable, implantable drug delivery device, and process for preparing and using same, U.S. Pat. No. 4,351,337).

By using the pharmaceutically acceptable compositions in accordance with the inventive methods, the symptoms of endometriosis are reduced or prevented, while simultaneously avoiding the side effects associated with other known medical treatments of endometriosis.

The present invention is also directed to a kit for the treatment and/or prevention of endometriosis. The kit is useful for practicing the inventive methods of treating and/or preventing endometriosis. The kit is an assemblage of materials or components, including at least one aryl hydrocarbon receptor binding ligand, as described above.

Instructions for using the AhR binding ligand in the inventive methods are also included in the kit. “Instructions for use” typically include a tangible expression describing the reagent concentration or at least one treatment method parameter, such as the relative amounts of reagents to be admixed, maintenance time periods for reagent admixtures, temperature, buffer conditions, administration method, dose, or dosing frequency, or the like, typically for an intended purpose.

Optionally, the kit also contains other useful components, such as, diluents, buffers, pharmaceutically acceptable carriers, syringes, stents, catheters, or pipetting or measuring tools.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures.

The components of the kit are typically contained in suitable packaging material(s). As employed herein, the phrase “packaging material” refers to one or more physical structures used to house the contents of the kit. The packaging material is constructed by well known methods, preferably to provide a sterile, contaminant-free environment.

The packaging materials employed in the kit are those customarily utilized in pharmaceutical systems. As used herein, the term “package” refers to a suitable solid matrix or material such as glass, plastic, paper, cardboard, foil, and the like, capable of holding the individual kit components. Thus, for example, a package can be a glass vial used to contain suitable quantities of the aryl hydrocarbon receptor binding ligands. The packaging material generally has an external label which indicates the contents and/or purpose of the kit and/or its components.

While the invention has been described with reference to its preferred embodiments, it will be appreciated by those skilled in this art that variations may be made departing from the precise examples of the methods and compositions disclosed herein, which, nonetheless, embody the invention defined by the following claims.

### EXAMPLES

### Example 1

### Animal Model of Endometriosis

Numerous animal models of endometriosis have been developed involving rabbits (Rock, J A et al., Intraocular endometrium in the rabbit as a model for endometriosis, Fertil. Steril. 59:232-35 [1993]), rodents (Sakura, Y et al., Histological studies on the therapeutic effect of sustained-release microspheres of a potent LHRH agonist (leuprorelin acetate) in an experimental endometriosis model in rats, Endocrinol. Jpn. 37:719-729 [1990]; Rajkumar, K et al., The rat as an animal model for endometriosis to examine recurrence of ectopic endometrial tissue after regression, Fertil. Steril. 53:921-25 [1990]; Sudo, K et al., Effects of TAP-144-SR, a sustained-release formulation of a potent GnRH agonist, on experimental endometriosis in the rat, Endocrinol. Jpn. 38:39-45 [1991]; Simms, J S et al., Identification of epidermal growth factor, transforming growth factor-α, and epidermal growth factor receptor in surgically induced endometriosis in rats, Obstet. Gynecol. 78(5 Part 1):850-57 [1991]; Tjaden, B et al., Time-released effects of RU-486 treatment in experimentally induced endometriosis in the rat, Fertil. Steril. 59:437-440 [1993]; Wright, J A. and Sharpe, K L, Adhesion formation in rats with surgically induced endometriosis, Contemporary Topics Lab. An. Sci. 33 :P15 [1994]; Cummings, A M and Metcalf, J L, Induction of endometriosis in mice: a new model sensitive to estrogen, Reprod. Toxicol. 9:233-38 [1995]; Cummings, A M et al., Promotion of endometriosis by 2,3,7,8-tetrachlorodibenzo-p-dioxin in rats and mice: time-dose dependence and species comparison, Toxicol. Appl. Pharmacol. 138:131-39 [1996]; Foster, W G et al., Morphologic characteristics of endometriosis in the mouse model: application to toxicology, Can. J. Physiol. Pharmacol. 75:1188-96 [1997]; Aoki, D et al., Successful heterotransplantation of human endometrium in SCID mice, Obstet. Gynecol. 83:220-28 [1994]; Bruner, K L et al., Suppression of matrix metalloproteinases inhibits establishment of ectopic lesions by human endometrium in nude mice, J. Clin. Invest. 99:2851-57 [1997]), and non-human primates (Schenken, R S et al., Etiology of infertility in monkeys with endometriosis: luteinized unruptured follicles, luteal phase defects, pelvic adhesions, and spontaneous abortions, Fertil. Steril. 41:122-30 [1984]; Rier, S E et al., Endometriosis in rhesus monkeys (Macaca mulatta) following chronic exposure to tetrachlorodibenzo-p-dioxin, Fund. Appl. Toxicol. 21:433-41 [1993]; D'Hooghe, T D et al., Development of a model of retrograde menstruation in baboons (Papio anubis), Fertil. Steril. 62:635-38 [1994]). Rodent models of endometriosis are of limited value because rodents do not spontaneously develop endometriosis, phylic differences in the estrous vs. menstrual cycle limit generalization of results to humans and transplanted endometrial fragments either subcutaneously or intra-abdominally in rodents do not fully resemble lesions seen in women. Therefore, a non-human primate model was selected, because monkeys and baboons spontaneously develop endometriosis, the pathophysiology of the disease resembles humans and abundant tissue can be obtained for study purposes. In particular, nulliparous female cynomolgus monkeys (Macaca fascicularis) were the animals chosen.

### Example 2

### Effects of the AhR Binding Ligand on Surgically Induced Endometriosis

The effects of subchronic exposure to an AhR binding ligand was examined in a non-human primate model. Endometrial fragments were surgically autotransplanted to the pelvic cavity of nulliparous cynomolgus monkeys (Macaca fascicularis, n=23). Briefly, each monkey's own endometrial tissue was obtained during a laparotomy in which the body of the uterus was opened in the anterior midline. A strip of endometrium was removed by careful dissection and divided into a number of equal pieces, one of which was used for histopathology to confirm the presence of both endometrial epithelium and stroma. The equal sized pieces were then transplanted into the abdomen/pelvis of the donor monkey in the following locations: periovarian, posterior uterine fundus, both broad ligaments. At one month post-transplantation a laparoscopy was performed and the implants were visualized, and a biopsy was performed to verify by histopathology that these lesions were indeed endometriotic implants.

The monkeys were divided into 4 treatment groups and dosed five days a week with gelatin capsules containing 0, 1, 5 or 25 ng/kg body mass of AhR binding ligand 2,3,7,8-tetrachlorodibenzo-p-dioxin (TCDD), mixed with glucose. Endometriotic implant survival and diameter were monitored by laparoscopy at intervals of one, three, six and 12 months.

Endometriotic lesion diameter was significantly reduced in the 0.71 ng/kg/day TCDD dose group compared to controls ([diameter in mm] 4.2±0.9 vs. 11.4±1.6, respectively) whereas the endometriotic lesion survival rate was unaffected (20% vs. 16%, respectively). In contrast, exposure to 3.57 and 17.86 ng/kg/day TCDD for one year resulted in a significantly higher survival rate of lesions (26.7% and 33.3% respectively vs. 16.0%) and significantly larger diameter lesions in the 17.86 ng/kg/day dose group only (14.5±0.2 vs. 11.4±1.6 mm in length) compared to the control group.

It is concluded that the AhR binding ligand exerted a bimodal effect on endometriotic tissue diameter at doses representative of human tissue levels. Endometriotic lesion diameter was reduced in the lowest TCDD dose group compared to the control group. These data show that AhR binding ligands can induce regression of endometriotic lesions.

We claim:

1. A method of treating endometriosis in a woman, comprising administering to a woman having endometriosis a pharmaceutically acceptable composition comprising:
at least one aryl hydrocarbon receptor binding ligand, in an amount effective to reduce the size of endometriotic tissue in the woman.

2. The method of claim 1, wherein the aryl hydrocarbon receptor binding ligand is indole-3-carbinol, indole-4-carbinol, or indole-5-carbinol.

3. The method of claim 1, wherein the aryl hydrocarbon receptor binding ligand is diindolylmethane.

4. The method of claim 1, wherein the aryl hydrocarbon receptor binding ligand is tryptophan, tryptamine, or indole acetic acid.

5. The method of claim 1, wherein the aryl hydrocarbon receptor binding ligand is a glucosinolate compound.

6. The method of claim 1, wherein the pharmaceutically acceptable composition is formulated as a food supplement.

7. The method of claim 1, wherein the aryl hydrocarbon receptor binding ligand is administered in an amount of about 1 milligram to about 10 grams per day.

8. The method of claim 1, wherein the aryl hydrocarbon receptor binding ligand is administered in an amount of about 1 gram to about 10 grams per day.

9. The method of claim 1, wherein the composition is administered through an oral delivery route.

10. The method of claim 9, wherein the oral delivery route is selected from the group consisting of ingestive and transmucosal delivery routes.

11. The method of claim 1, wherein the composition is administered through a parenteral delivery route.

12. The method of claim 11, wherein the parenteral delivery route is selected from the group consisting of intradermal, subcutaneous, intramuscular, intravenous, intravaginal, intranasal, intrabronchial, and rectal delivery routes.

13. A method of preventing endometriosis in a woman at higher than normal risk of developing endometriosis, comprising:
administering to the woman at higher than normal risk of developing endometriosis a pharmaceutically acceptable composition comprising at least one aryl hydrocarbon receptor binding ligand, in an amount effective to inhibit the growth or thickening of endometriotic tissue in the woman.

14. The method of claim 13, wherein the aryl hydrocarbon receptor binding ligand is indole-3-carbinol, indole-4-carbinol, or indole-5-carbinol.

15. The method of claim 13, wherein the aryl hydrocarbon receptor binding ligand is diindolylmethane.

16. The method of claim 13, wherein the aryl hydrocarbon receptor binding ligand is tryptophan, tryptamine, or indole acetic acid.

17. The method of claim 13, wherein the aryl hydrocarbon receptor binding ligand is a glucosinolate compound.

18. The method of claim 13, wherein the pharmaceutically acceptable composition is formulated as a food supplement.

19. The method of claim 13, wherein the aryl hydrocarbon receptor binding ligand is administered in an amount of about 1 milligram to about 10 grams per day.

20 The method of claim 13, wherein the aryl hydrocarbon receptor binding ligand is administered in an amount of about 1 gram to about 10 grams per day.

21. The method of claim 13, wherein the composition is administered through an oral delivery route.

22. The method of claim 20, wherein the oral delivery route is selected from the group consisting of ingestive and transmucosal.

23. The method of claim 13, wherein the composition is administered through a parental delivery route.

24. The method of claim 23, wherein the parenteral delivery route is selected from the group consisting of intradermal, subcutaneous, intramuscular, intravenous, intravaginal, intranasal, intrabronchial, and rectal.

25. A kit for treating and/or preventing endometriosis in a woman, comprising:
a pharmaceutically acceptable composition comprising an amount of at least one aryl hydrocarbon receptor binding ligand in a suitable package; and instructions for using the at least one aryl hydrocarbon receptor binding ligand to reduce the size of endometriotic tissue and/or to inhibit the growth or thickening of endometriotic tissue.

26. The kit of claim 25, wherein the aryl hydrocarbon receptor binding ligand is indole-3-carbinol, indole-4-carbinol, or indole-5-carbinol.

27. The kit of claim 25, wherein the aryl hydrocarbon receptor binding ligand is diindolylmethane.

28. The kit of claim 25, wherein the aryl hydrocarbon receptor binding ligand is tryptophan, tryptamine, or indole acetic acid.

29. The kit of claim 25, wherein the aryl hydrocarbon receptor binding ligand is a glucosinolate compound.

30. The kit of claim 25, wherein the pharmaceutically acceptable composition is formulated as a food supplement.

31. The kit of claim 25, wherein the amount of aryl hydrocarbon receptor binding ligand is in an amount of about 1 milligram to about 10 grams.

32. The kit of claim 25, wherein the amount of aryl hydrocarbon receptor binding ligand is in an amount of about 1 gram to about 10 grams.

33. The kit of claim 25, wherein the composition is formulated for an oral delivery route.

34. The kit of claim 33, wherein the oral delivery route is an ingestive delivery route.

35. The kit of claim 33, wherein the oral delivery system is a transmucosal delivery route.

36. The kit of claim 25, wherein the composition is formulated for a parental delivery route.

37. The kit of claim 36, wherein the parenteral delivery route is selected from the group consisting of intradermal, subcutaneous, intramuscular, intravenous, intravaginal, intranasal, intrabronchial, and rectal delivery routes.

38. The kit of claim 25, wherein the composition is formulated for a delivery system selected from the group consisting of capsules, caplets, tablets, microspheres, powders, suspensions, emulsions, ingestible solutions, syrups, aerosols, suppositories, and injectable solutions.

39. Use of an aryl hydrocarbon receptor binding ligand in the manufacture of a medicament for inhibiting the growth of and/or reducing the size of endometriotic tissues.

40. The use of claim 39, wherein the aryl hydrocarbon receptor binding ligand is indole-3-carbinol, indole-4-carbinol, or indole-5-carbinol.

41. The use of claim 39, wherein the aryl hydrocarbon receptor binding ligand is diindolylmethane.

42. The use of claim 39, wherein the aryl hydrocarbon receptor binding ligand is tryptophan, tryptamine, or indole acetic acid.

43. The use of claim 39, wherein the aryl hydrocarbon receptor binding ligand is a glucosinolate compound.
